(19) United States
(12) Patent Application Publication
(10) Pub. No.: US 2005/0036970 A1
(43) Pub. Date: Feb. 17, 2005

(54) Reducing compositions for bleaching or permanently reshaping keratin fibres comprising polycarboxylic acids and salts thereof as complexing agents

(75) Inventors: Anne Sabbagh, Rueil Malmaison (FR); Frederic Legrand, Courbevoie (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL S.A., Paris (FR)

(21) Appl. No.: 10/809,879

(22) Filed: Mar. 25, 2004

Related U.S. Application Data
(60) Provisional application No. 60/461,982, filed on Apr. 11, 2003.

(30) Foreign Application Priority Data
Mar. 28, 2003 (FR) 0350079
Mar. 25, 2003 (FR) 0350066

(51) Int. Cl. A61K 7/09

(52) U.S. Cl. 424/70.2

(57) ABSTRACT

The present invention relates to a ready-to-use reducing composition for bleaching or permanently reshaping keratin fibres. The reducing composition comprises polycarboxylic acids and salts thereof as completing agents. The invention also relates to a method of using a reducing composition and a kit for bleaching or permanently reshaping keratin fibres.

### TECHNICAL FIELD

The present invention relates to reducing compositions for bleaching or permanently reshaping keratin fibres, which comprise polycarboxylic acids and salts thereof as complexing agents.

The invention also relates to processes and devices or “kits” for bleaching or permanently reshaping keratin fibres, and also to the use of these compositions, processes and kits for bleaching or permanently reshaping human keratin fibres and more especially the hair.

### PRIOR ART

Two types of composition are used to bleach keratin fibres: compositions that are, “oxidizing”, since they contain one or more agents capable of oxidizing the melanin of the hair and thus of dissolving it in order to totally or partially remove it, and compositions that are, in contrast, “reducing”, since they contain one or more reducing agents such as ascorbic acid, sulphites or sulphinates, and that are intended more especially for bleaching hair that has previously been dyed with exogenous pigments.

Moreover, for permanently reshaping the hair, it is common practice to apply to hair that has been placed under tension beforehand, for example using rollers if the desired reshaping is curls, a composition containing one or more reducing agents so as to induce the opening of the disulphide bridges formed by the cysteine residues of the hair keratin, and then, generally after rinsing, to reoxidize the hair in order to fix its reshaping.

The reducing agents preferably used in the context of the permanent reshaping of the hair are thiols such as thioglycolic acid and thiolactic acid, and the salts and esters thereof, and sulphites.

Whether they are intended for bleaching or permanent reshaping, the reducing compositions contain, in principle, an agent intended to complex the metal cations that may be present in trace amounts in these compositions, and also those that may be present on the hair, originating from the ambient air, from the water with which the hair has been washed, or shampoos or other hair products with which the hair has been treated.

Specifically, it is very important to neutralize these metal cations, since they are capable of catalysing oxidation reactions, and of doing so in an uncontrolled manner, which may be reflected by severe adverse effects such as breaking of the hair or burning of the scalp.

The complexing agents that are currently the most commonly used in reducing compositions for bleaching or permanently reshaping keratin fibres are ethylenediaminetetraacetic acid (EDTA) and derivatives thereof, for instance diethylenetriaminepentaacetic acid (DPTA), generally in weight proportions of about 0.1% to 1%.

However, in the context of its research, the Applicant has found that EDTA and derivatives thereof have insufficient properties in compositions of this type. These findings, which are corroborated by the results obtained by other research teams, justify the search for novel complexing agents.

A complexing agent intended to form part of the constitution of reducing compositions for bleaching or permanently reshaping keratin fibres should satisfy many requirements.

Specifically, besides the fact that it should have high complexing power with respect to metals so as to remove, or at the very least minimize, the risk of catalysis of the oxidation reaction on the keratin fibres by the metals possibly present, even as traces, in these compositions and on these fibres, it should be compatible, and especially should not react, with the other constituents of these compositions, and in particular with the reducing agent(s).

It should also be stable in an aqueous medium, since, specifically, the reducing compositions are generally applied to the keratin fibres in the form of aqueous compositions.

It should also be entirely harmless with respect to these fibres and to the skin, and especially should have no allergenic nature.

It is also desirable, out of concern for the environment, for it to be biodegradable, and for its production cost or purchase cost to allow it to be used in compositions intended to be sold not only to professionals but also in mass distribution.

Now, after extensive research conducted in this matter, the Applicant has found that, surprisingly, certain polycarboxylic acids and salts thereof are capable of satisfying all these requirements and consequently of representing complexing agents of choice in reducing compositions for dyeing, bleaching and permanently reshaping keratin fibres.

This finding forms the basis of the invention.

### DESCRIPTION OF THE INVENTION

A first subject of the invention is a reducing composition for bleaching or permanently reshaping keratin fibres, comprising at least one reducing agent, said composition being characterized in that it comprises at least one compound corresponding to the general formula (I) below:
R—N—(CH(R′)CO₂X)₂ (I)
in which:
R represents a hydrogen atom or a group —CH(CO₂X)—(CH₂)₂CO₂X, —CH₂—CH₂—OH, —CH(CH₃)—CO₂X or —(CH₂)₂—N(COR″)—CH₂—CO₂X;
R″ represents a linear or branched alkyl group containing from 1 to 30 carbon atoms, or a cycloalkyl group containing from 3 to 30 carbon atoms;
R′ represents a group —CH₂CO₂X when R represents a hydrogen atom, whereas R′ represents a hydrogen atom when R is other than a hydrogen atom; and
X represents a hydrogen atom or a monovalent or divalent cation derived from an alkali metal, from an alkaline-earth metal, from a transition metal or from an organic amine, or an ammonium cation.

Thus, the complexing agents used in the context of the invention correspond to polycarboxylic acid compounds and to the corresponding carboxylates.

More specifically, the complexing agents correspond to:
compounds comprising four carboxylic acid or carboxylate functions, when R represents a hydrogen atom and R′ represents a group —CH₂—CO₂X, or when R represents a group —CH(CO₂X)—(CH₂)₂—CO₂X and R′ represents a hydrogen atom;
compounds comprising three carboxylic acid or carboxylate functions, when R represents a group —CH(CH₃)—CO₂X and R′ represents a hydrogen atom, or when R represents a group —(CH₂)₂—N(COR″)—CH₂—CO₂X and R′ represents a hydrogen atom; and to
compounds comprising two carboxylic acid or carboxylate functions, when R represents the group —CH₂—CH₂—OH and R′ represents a hydrogen atom.

In accordance with the invention, when the compound(s) of formula (I) is(are) carboxylates, then the monovalent or divalent cation is preferably chosen from the group consisting of monovalent alkali metal cations, divalent alkaline-earth metal cations, divalent transition metal cations and monovalent cations derived from organic amines or from ammonium.

Examples of alkali metal cations that may especially be mentioned include sodium (Na⁺) and potassium (K⁺), while examples of alkaline-earth metal cations that may especially be mentioned include calcium (Ca²⁺) and magnesium (Mg²⁺).

For the purpose of the present invention, the term “transition metal” means a metal comprising an incomplete d subshell, more particularly in oxidation state II, such as cobalt (Co²⁺), iron (Fe²⁺), manganese (Mn²⁺), zinc (Zn²⁺) and copper (Cu²⁺).

As regards the organic amine cations, mention may be made of primary, secondary or tertiary amine cations or alternatively alkanolamine cations.

Said amines contain one or more radicals, which may be identical or different, of linear or branched C₁ to C₂₀ alkyl type, optionally comprising a heteroatom such as oxygen.

As regards the quaternary ammonium cations, these comprise three radicals, which may be identical or different, chosen from hydrogen and a linear or branched C₁ to C₂₀ alkyl radical, optionally comprising a heteroatom such as oxygen.

In accordance with the invention, when the compound(s) of formula (I) is (are) carboxylates, then the monovalent or divalent cation is preferably chosen from the group consisting of alkali metal cations, alkaline-earth metal cations, divalent transition metal cations, and cations derived from organic amines or from ammonium.

The compound(s) of formula (I) is (are) preferably chosen from the group consisting of methylglycine diacetic acid, 2-hydroxyethyliminodiacetic acid, N-lauroyl-N,N′,N′-ethylenediamine triacetic acid, iminodisuccinic acid and N,N-dicarboxymethyl-L-glutamic acid, the alkali metal salts thereof, the alkaline-earth metal salts thereof, the transition metal salts thereof, the organic amine salts thereof and the ammonium salts thereof, and mixtures thereof.

Methylglycine diacetic acid, 2-hydroxyethyliminodiacetic acid, N-lauroyl-N,N′,N′-ethylenediamine triacetic acid, iminodisuccinic acid and N,N-dicarboxymethyl-L-glutamic acid and, and the salts thereof, are respectively represented by formulae (II), (III), (IV), (V) and (VI) below: 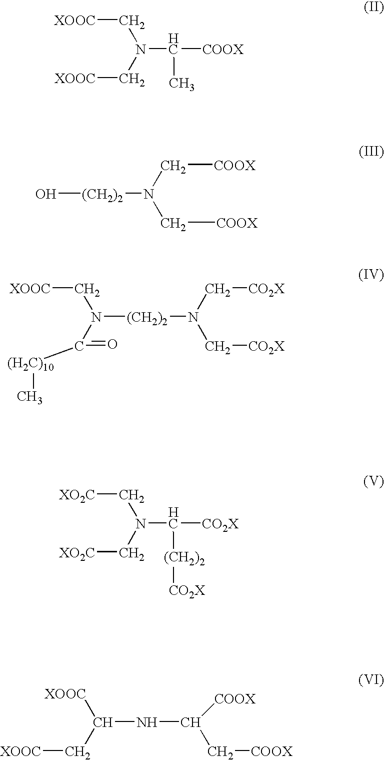
in which X is as defined above, X preferably corresponding to H or Na.

These compounds are especially available from the companies BASF, Dow Chemical, Hampshire, Bayer and Showa Denko.

2-Hydroxyethyliminodiacetic acid, methylglycine diacetic acid, the sodium salts thereof and the mixtures thereof are more particularly preferred.

Preferably, the compound(s) of formula (I) represent(s) from 0.001% to 10% by weight and better still from 0.001% to 5% by weight relative to the total weight of the reducing composition. The weight percentages are expressed relative to the acid form of the compound(s) of formula (I).

In accordance with the invention, the reducing agent(s) present in the reducing composition may be chosen without preference from any reducing agent whose use in the field of bleaching and permanently reshaping keratin fibres has already been proposed.

However, in the case of a composition intended for bleaching, this or these reducing agent(s) is(are) preferably chosen from the group consisting of reductones such as ascorbic acid and erythorbic acid, and the salts and esters thereof, sulphites, for instance sodium sulphite, and sulphinates, for instance sodium hydroxymethane sulfinate, whereas, in the case of a composition intended for permanent reshaping, it is preferred to use one or more thiols, and in particular thioglycolic acid, thiolactic acid, cysteamine and cysteine, and the salts and esters thereof, and/or one or more sulphites or sulphinates.

It is recalled that reductones are lactones of reductive nature.

In all cases, the reducing agent(s) preferably represent(s) from 0.1% to 30% by weight and better still from 0.5% to 20% by weight relative to the total weight of the reducing composition.

Preferably, the reducing composition comprises, in addition to the compound(s) of formula (I) and the reducing agent(s), one or more compounds chosen from: (A) cationic or amphoteric conditioning polymers, (B) nonionic, anionic, cationic or amphoteric amphiphilic polymers comprising a hydrophobic chain, (C) surfactants, (D) rheology modifiers other than the polymers (B), (E) pH modifiers, and/or (F) solvents.

(A) Cationic or Amphoteric Conditioning Polymers:

For the purposes of the present invention, the term “cationic conditioning polymer” means any polymer which comprises cationic groups or groups that can be ionized into cationic groups and which can improve the cosmetic properties of keratin fibres, in particular the disentangling, the softness, the sheen and the volume.

The cationic or amphoteric conditioning polymers that are suitable are advantageously chosen from those already known per se as improving the cosmetic properties of the hair, that is to say, especially, those described in patents and patent applications EP 337 354, FR 2 270 846, FR 2 383 660, FR 2 598 611, FR 2 470 596, FR 2 519 863, FR 2 788 974 and FR 2 788 976.

However, more specific examples of cationic conditioning polymers that may especially be mentioned include cationic polymers comprising at least primary, secondary, tertiary and/or quaternary amine groups, which either may form part of the main polymer chain, or may be borne by a side substituent directly attached thereto.

Thus, mention may be made of:

(1) copolymers of acrylamide and of dimethylaminoethyl methacrylate quaternized with dimethyl sulphate or with a dimethyl halide (Hercofloc from Hercules); copolymers of acrylamide and of methacryloyloxyethyltrimethylammonium chloride (Bina Quat P 100 from Ciba Geigy); the copolymer of acrylamide and of methacryloyloxyethyltrimethylammonium methosulphate (Reten from Hercules); quaternized or non-quaternized vinylpyrrolidone/dialkylaminoalkyl acrylate or methacrylate copolymers (Gafquat range from ISP; Copolymer 845, 958 and 937 from ISP); dimethylaminoethyl methacrylate/vinylcaprolactam/vinylpyrrolidone terpolymers (Gaffix VC 713 from ISP); vinylpyrrolidone/methacrylamidopropyldimethylamine copolymers (Styleze CC 10 from ISP); vinylpyrrolidone/dimethylaminopropylmethacrylamide quaternized copolymers (Gafquat HS 100 from ISP);

(2) Cellulose ether derivatives comprising quaternary ammonium groups, as described in FR 1 492 597. These polymers are also defined in the CTFA dictionary as quaternary ammoniums of hydroxyethylcellulose that has reacted with an epoxide substituted with a trimethylammonium group;

(3) Cationic cellulose derivatives such as copolymers of cellulose or cellulose derivatives grafted with a water-soluble quaternary ammonium monomer, described especially in U.S. Pat. No. 4,131,576, such as hydroxyalkylcelluloses, for instance hydroxymethylcellulose, hydroxyethylcellulose or hydroxypropylcellulose grafted especially with a methacryloylethyltrimethylammonium, methacrylamidopropyltrimethylammonium or dimethyldiallylammonium salt;

(4) The cationic polysaccharides described more particularly in patents U.S. Pat. No. 3,589,578 and U.S. Pat. No. 4,031,307, such as guar gums containing trialkylammonium cationic groups. Guar gums modified with a salt, for instance the chloride, especially 2,3-epoxy-propyltrimethylammonium chloride, are used for example;

(5) Polymers consisting of piperazinyl units and of divalent alkylene or hydroxyalkylene radicals containing straight or branched chains, optionally interrupted with oxygen, sulphur or nitrogen atoms or with aromatic or heterocyclic groups, and also the oxidation and/or quaternization products of these polymers. Such polymers are described especially in FR 2 162 025 and FR 2 280 361;

(6) Water-soluble polyaminoamides prepared in particular by polycondensation of an acidic compound with a polyamine, which are optionally crosslinked, optionally alkylated, or, if they comprise one or more tertiary amine functions, quaternized. These polymers are described especially in FR 2 252 840 and FR 2 368 508;

(7) Polyaminoamide derivatives resulting from the condensation of polyalkylene polyamines with polycarboxylic acids followed by an alkylation with difunctional agents. Examples that may be mentioned include adipic acid-dialkylaminohydroxyalkyldialkylene-triamine polymers in which the alkyl radical is C₁-C₄. Such polymers are described especially in FR 1 583 363;

(8) Polymers obtained by reacting a polyalkylene polyamine comprising two primary amine groups and at least one secondary amine group with a dicarboxylic acid chosen from diglycolic acid and saturated C₃-C₈ aliphatic dicarboxylic acids, and then with epichlorohydrin. Such polymers are described especially in U.S. Pat. No. 3,227,615 and U.S. Pat. No. 2,961,347;

(9) Cyclopolymers of alkyldiallylamine or of dialkyldiallylammonium, in homopolymer or copolymer form, as described in FR 2 080 759 and in its Certificate of Addition No. 2 190 406;

(10) Diquaternary ammonium polymers as described in FR 2 320 330, FR 2 270 846, FR 2 316 271, FR 2 336 434, FR 2 413 907, U.S. Pat. No. 2,273,780, U.S. Pat. No. 2,375,853, U.S. Pat. No. 2,388,614, U.S. Pat. No. 2,454,547, U.S. Pat. No. 3,206,462, U.S. Pat. No. 2,261,002, U.S. Pat. No. 2,271,378, U.S. Pat. No. 3,874,870, U.S. Pat. No. 4,001,432, U.S. Pat. No. 3,929,990, U.S. Pat. No. 3,966,904, U.S. Pat. No. 4,005,193, U.S. Pat. No. 4,025,617, U.S. Pat. No. 4,025,627, U.S. Pat. No. 4,025,653, U.S. Pat. No. 4,026,945 and U.S. Pat. No. 4,027,020; mention may be made, for example, of polymers comprising the following repeating units: 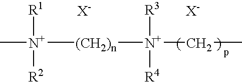
in which the radicals R¹, R², R³ and R⁴ which may be identical or different, denote a C₁-C₄ alkyl or hydroxyalkyl radical, n and p are integers ranging from 2 to 20 and X⁻ is an anion derived from a mineral or organic acid;

(11) Poly(quaternary ammonium) polymers consisting of repeating units of formula: 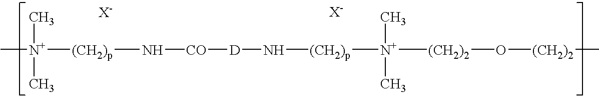
in which p denotes an integer ranging from 1 to 6 approximately, D may be nothing or may represent a group —(CH₂)ᵣ—CO— in which r denotes a number equal to 4 or to 7, and X⁻ is an anion. Such polymers may be prepared according to the processes described in U.S. Pat. No. 4,157,388, U.S. Pat. No. 4,702,906, U.S. Pat. No. 4,719,282 and EP 122 324;

(12) Quaternary polymers of vinylpyrrolidone and of vinylimidazole;

(13) Polyamines of the polyethylene glycol (15) tallow polyamine type (CTFA dictionary name);

(14) Crosslinked methacryloyloxy (C₁-C₄) alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. A crosslinked acrylamide/methacryloyloxyethyltrimethyl-ammonium chloride copolymer (20/80 by weight) in the form of a dispersion containing 50% by weight of the said copolymer in mineral oil (Salcare® SC 92 from Ciba) can be used more particularly. A crosslinked methacryloyloxyethyltrimethylammonium chloride homopolymer containing about 50% by weight of the homopolymer in mineral oil or in a liquid ester (Salcare® SC 95, SC 96 from Ciba) can also be used.

Other cationic conditioning polymers that can be used in the context of the invention are polyalkyleneimines, in particular polyethyleneimines, polymers containing vinylpyridine or vinylpyridinium units, condensates of polyamines and of epichlorohydrin, quaternary polyureylenes and chitin derivatives.

The amphoteric conditioning polymer(s) that may be present in the reducing composition may themselves be chosen especially from: polymers comprising units K and M randomly distributed in the polymer chain, in which K denotes a unit derived from a monomer comprising at least one basic nitrogen atom and M denotes a unit derived from an acidic monomer comprising one or more carboxylic or sulphonic groups; alternatively K and M may denote groups derived from zwitterionic carboxybetaine or sulphobetaine monomers; alternatively, K and M denote a cationic polymer chain comprising primary, secondary, tertiary or quaternary amine groups, in which at least one of the amine groups bears a carboxylic or sulphonic group linked via a hydrocarbon-based radical; alternatively K and M form part of a chain of a polymer containing an α,β-dicarboxylic ethylene unit in which one of the carboxylic groups has reacted with a polyamine comprising one or more primary or secondary amine groups.

The amphoteric conditioning polymers corresponding to the above definition that are more particularly preferred are chosen from the following polymers:

(1) polymers resulting from the copolymerization of a monomer derived from a vinyl compound bearing a carboxylic group such as, more particularly, (meth)acrylic acid, maleic acid, α-chloroacrylic acid, or else a dialkyldiallylammonium salt such as dimethyldiallylammonium chloride, and a basic monomer derived from a substituted vinyl compound containing at least one basic atom, such as, more particularly, dialkylaminoalkyl methacrylate and acrylate, dialkylaminoalkylmethacrylamide and -acrylamide, as described in U.S. Pat. No. 3,836,537. Mention may also be made of the sodium acrylate/acrylamidopropyl-trimethylammonium chloride copolymer (Polyquart KE 3033 from Cognis) and the acrylic acid/dimethyldiallyl-ammonium chloride copolymer (Merquat 280, 295, Plus 3330, from Nalco);

(2) polymers containing units derived from: a) at least one monomer chosen from (meth)acrylamides substituted on the nitrogen with an alkyl radical, in particular C₂-C₁₂, b) at least one acidic monomer containing one or more reactive carboxylic groups (for example (meth)acrylic acid, crotonic acid or itaconic acid, and monoesters of maleic or fumaric acids or anhydrides), and c) at least one basic monomer such as esters containing primary, secondary, tertiary and quaternary amine substituents of (meth)acrylic acid, fumaric acid or maleic acid, and the product of quaternization of dimethylaminoethyl methacrylate with dimethyl or diethyl sulphate. Octylacrylamide/acrylate/butylaminoethyl methacrylate copolymers (Amphomer or Lovocryl 47 by the company National Starch) are particularly used ;

(3) Crosslinked and partially or totally alkylated polyaminoamides, derived from polyaminoamides of general formula —[CO—R⁵—CO-Z]- in which R⁵ is a divalent radical derived from a saturated or unsaturated dicarboxylic acid (for example adipic acid, 2,2,4-trimethyladipic acid, 2,4,4-trimethyladipic acid, terephthalic acid or itaconic acid), from an unsaturated monocarboxylic acid (for instance (meth)acrylic acid), from a C₁-C₆ alkyl ester of the abovementioned acids or from a radical derived from the addition of one of these acids to a bis-primary or bis-secondary amine, and Z denotes a radical of a bis-primary, mono- or bis-secondary polyalkylene-polyamine. Preferably, Z represents between 60 and 100 mol %, the radical —NH—[(CH₂)ₓ—NH]ₚ— with x=2 and p=2 or 3, or x=3 and p=2; this radical is derived from diethylenetriamine, from triethylenetetramine or from dipropylenetriamine; between 0 and 40 mol % the above radical, in which x=2 and p=1 and which is derived from ethylenediamine, or the radical derived from piperazine —N[CH₂CH₂]₂N—; between 0 and 20 mol %, the radical —NH—(CH₂)₆—NH— derived from hexamethylenediamine. The crosslinking agent for these polymers is a difunctional agent chosen from epihalohydrins, diepoxides, dianhydrides and bis-unsaturated derivatives, and alkylated by the action of acrylic acid, chloroacetic acid or an alkane sulfone or the alkali metal salts thereof;

(4) Polymers comprising at least zwitterionic units, for instance the butyl methacrylate/dimethylcarboxymethylammonioethyl methacrylate copolymer (Diaformer Z301 from Sandoz);

(5) Polymers derived from chitosan comprising monomer units corresponding to formulae (I), (II) and (III) below: 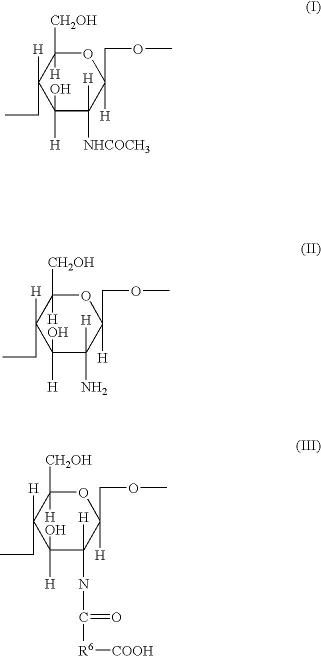
with (I) representing from 0 to 30%, (II) from 5% to 50% and (III) from 30% to 90% in which R⁶ represents a radical of formula: 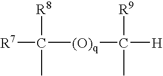
in which q denotes 0 or 1; and if q=0, R⁷, R⁸ and R⁹, which may be identical or different, represent a hydrogen, a methyl, hydroxyl, acetoxy, amino, monoalkylamino or dialkylamino group, optionally interrupted with one or more nitrogen atoms and/or optionally substituted with one or more amine, hydroxyl or carboxyl groups, alkylthio groups optionally bearing an amino group, or sulphonic group; or, if q=1, R⁷, R⁸ and R⁹, which may be identical or different, represent a hydrogen, and also the salts formed by these compounds with acids or bases;

(6) Polymers derived from the N-carboxyalkylation of chitosan, for instance N-carboxymethylchitosan or N-carboxybutylchitosan (Evalsan from Jan Dekker);

(7) Polymers as described in FR 1 400 366: 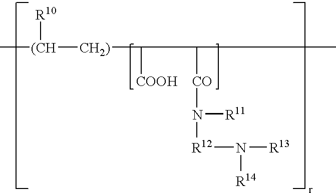
in which R¹⁰ is a hydrogen, CH₃O—, CH₃CH₂O— or phenyl, R¹¹ and R¹⁴, which may be identical or different, represent a hydrogen or an alkyl radical (methyl or ethyl), R¹³ represents an alkyl radical (methyl or ethyl) or a radical of formula —R¹²—N(R¹⁴)₂, R¹² representing —(CH₂)₂—, —(CH₂)₃— or —CH₂—CH(CH₃)— and also the higher homologues of these radicals and containing up to 6 carbon atoms, and r is such that the molecular weight is between 500 and 6 000 000 and preferably between 1 000 and 1 000 000;

(8) Amphoteric polymers of the type -D¹-X-D¹-X— chosen from:

a) polymers obtained by the action of chloroacetic acid or sodium chloroacetate on compounds comprising at least one unit of formula -D¹-X-D¹-X-D¹- in which D¹ denotes a piperazinyl radical and X denotes the symbol E or E′, E or E′, which may be identical or different, denote a divalent radical which is an alkylene radical containing a straight or branched chain containing up to 7 carbon atoms in the main chain, optionally substituted with hydroxyl groups and possibly also comprising oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;

b) polymers of formula -D¹-X-D¹-X— in which D¹ denotes a piperazinyl radical and X denotes the symbol E or E′ and at least once E′; E having the meaning given above and E′ being a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate;

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

Among the cationic or amphoteric conditioning polymers that may be used, the following are especially preferred:

(i) among the cationic polymers:
the dimethyldiallylammonium chloride homopolymer (Merquat 100 from Nalco);
copolymers of dimethyldiallylammonium chloride and of acrylamide (Merquat 2200 from Nalco);
polymers of poly(quaternary ammonium) type prepared and described in FR 2 270 846, consisting of repeating units of formulae (W) and (U) below: 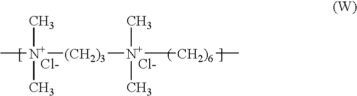
   and especially those with a molecular weight, determined by gel permeation chromatography, of between 9 500 and 9 900; 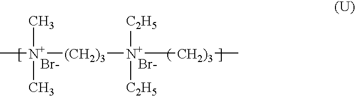
   and especially those with a molecular weight, determined by gel permeation chromatography, of about 1 200;
polymers of poly(quaternary ammonium) type of the family (11) with X⁻ denoting chlorine, and especially those with a weight-average molecular mass of less than 100 000 and preferably less than or equal to 50 000;

(ii) among the amphoteric polymers:
dimethyldiallylammonium chloride/acrylic acid copolymer (80/20) (Merquat 280 from Nalco—CTFA name: Polyquaternium 22);
dimethyldiallylammonium chloride/acrylic acid copolymer (95/5) (Merquat 295 from Nalco);
methacrylamidopropyltrimonium chloride, acrylic acid and ethyl acrylate copolymer (Merquat 2001 from Nalco—CTFA name: Polyquaternium 47);
acrylamide/dimethyldiallylammonium chloride/acrylic acid terpolymer (Merquat Plus 3330 from Nalco—CTFA name: Polyquaternium 39).

When the reducing composition comprises one or more cationic or amphoteric conditioning polymers, this or these polymer(s) then generally represent from 0.01% to 10% by weight and preferably from 0.05% to 5% of the total weight of this composition.

(B) Nonionic, Anionic, Cationic or Amphoteric Amphiphilic Polymers Comprising a Hydrophobic Chain:

More particularly, the hydrophobic chain is a saturated or unsaturated, aromatic or non-aromatic, linear or branched C₆-C₃₀ hydrocarbon-based chain, optionally comprising one or more oxyalkylene (oxyethylene and/or oxypropylene) units.

Among the cationic amphiphilic polymers comprising a hydrophobic chain that may be found are cationic polyurethanes or cationic copolymers comprising vinyllactam units and in particular vinylpyrrolidone units.

Preferably, the amphiphilic polymers comprising a hydrophobic chain will be of nonionic or anionic nature.

As examples of nonionic amphiphilic polymers containing a hydrophobic chain, mention may be made, inter alia, of:

(1) celluloses modified with groups comprising at least one saturated or unsaturated, linear or branched C₆-C₃₀ hydrocarbon-based chain, for instance hydroxyethylcelluloses modified with groups comprising at least one hydrophobic chain as defined previously, such as especially Natrosol Plus Grade 330 CS (C₁₆ alkyls—sold by the company Aqualon); Bermocoll EHM 100 (sold by the company Berol Nobel), Amercell Polymer HM-1500 (hydroxyethylcellulose modified with a polyethylene glycol (15) nonylphenyl ether group—sold by the company Amerchol);

(2) hydroxypropyl guars modified with groups comprising at least one hydrophobic chain as defined, for example Jaguar XC-95/3 (C₁₄ alkyl chain—sold by the company Rhodia Chimie); Esaflor HM 22 (C₂₂ alkyl chain—sold by the company Lamberti); RE210-18 (C₁₄ alkyl chain) and RE205-1 (C₂₀ alkyl chain) sold by the company Rhodia Chimie;

(3) copolymers of vinylpyrrolidone and of hydrophobic monomers containing a hydrophobic chain as defined above, for instance Antaron or Ganex V216 (vinylpyrrolidone/hexadecene copolymers); Antaron or Ganex V220 (vinylpyrrolidone/eicosene copolymers), sold by the company I.S.P.;

(4) copolymers of C₁-C₆ alkyl (meth)acrylates and of amphiphilic monomers containing a hydrophobic chain;

(5) copolymers of hydrophilic (meth)acrylates and of hydrophobic monomers comprising at least one hydrophobic chain, for instance the polyethylene glycol methacrylate/lauryl methacrylate copolymer;

(6) polymers with an aminoplast ether skeleton containing at least one fatty chain, such as the Pure Thix compounds sold by the company Süd-Chemie;

(7) linear (block structure), grafted or starburst polyurethane polyethers comprising in their chain at least one hydrophilic block, which is generally a polyoxyethylene block which may comprise between 50 and 1 000 oxyethylene units approximately, and at least one hydrophobic block, which may comprise aliphatic groups alone, optionally combined with cycloaliphatic and/or aromatic blocks. Preferably, the polyurethane polyethers comprise at least two C₆-C₃₀ hydrocarbon-based hydrophobic chains, separated by a hydrophilic block; the hydrophobic chains may be pendent chains or chains with one or more of the end groups of the hydrophilic block(s).

The polyurethane polyethers comprise a urethane bond between the hydrophilic blocks, but may also contain hydrophilic blocks linked to the lipophilic blocks via other chemical bonds.

The polyurethane polyethers are in particular those described in the article by G. Fonnum, J. Bakke and Fk. Hansen—Colloid Polym. Sci. 271, 380-389 (1993). Examples of polyurethane polyethers that may be mentioned include Nuvis FX 1100 (European and US INCI name “Steareth-100/PEG-136/HMDI Copolymer” sold by the company Servo Delden); Rheolate 205, 208, 204 or 212 (sold by the company Rheox); Elfacos T210 (C₁₂-C₁₄ alkyl chain) and Elfacos T212 (C₁₈ alkyl chain) sold by the company Akzo.

The anionic amphiphilic polymers containing a hydrophobic chain that may be used comprise, as hydrophobic chain, at least one saturated or unsaturated, aromatic or non-aromatic, linear or branched C₈-C₃₀ hydrocarbon-based chain.

More particularly, the anionic amphiphilic polymers comprising at least one hydrophobic chain which are crosslinked or non-crosslinked, comprise at least one hydrophilic unit derived from one or more ethylenically unsaturated monomers bearing a carboxylic acid function, or a sulphonic function which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

Anionic amphiphilic polymers of the type described above are described and prepared, for example, in patents U.S. Pat. No. 3,915,921 and U.S. Pat. No. 4,509,949 (copolymers of (meth)acrylic acid and of C₁₀-C₃₀ alkyl (meth)acrylates) or in patent EP 216 479 (copolymers of (meth)acrylic acid and of fatty alkyl allyl ethers).

The amphiphilic polymers comprising at least one sulphonic group, in free or partially or totally neutralized form and at least one hydrophobic portion are described, for example, in FR 00 16954 and FR 01 00328, the content of which forms an integral part of the present invention.

Among these, mention may be made more particularly of acrylamido-2-methyl-2-propanesulphonic (AMPS) acid/n-dodecylacrylamide copolymer neutralized with sodium hydroxide, the copolymer crosslinked with methylenebisacrylamide consisting of 75% by weight of AMPS units neutralized by NH₃ and 25% by weight of Genapol T-250 acrylate units, the copolymer crosslinked with allyl methacrylate consisting of 90% by weight of AMPS units neutralized with NH₃ and 10% by weight of Genapol T-250 methacrylate units, or the copolymer crosslinked with allyl methacrylate consisting of 80% by weight of AMPS units neutralized with NH₃ and 20% by weight of Genapol T-250 methacrylate units.

Examples of preferred polymers that may be mentioned include Carbopol ETD-2020 (acrylic acid/C₁₀-C₃₀ alkyl methacrylate crosslinked copolymer—sold by the company Noveon); Carbopol 1382, Pemulen TR1 and Pemulen TR2 (acrylic acid/C₁₀-C₃₀ alkyl acrylate crosslinked copolymers—sold by the company Noveon), the methacrylic acid/ethyl acrylate/oxyethylenated stearyl methacrylate copolymer (55/35/10); the (meth)acrylic acid/ethyl acrylate/25 EO oxyethylenated behenyl methacrylate copolymer (Aculyn 28 sold by Rohm & Haas) and the methacrylic acid/ethyl acrylate/steareth-10 allyl ether crosslinked copolymer.

When the reducing composition comprises one or more amphiphilic polymer(s) containing a fatty chain, then this or these polymer(s) generally represent(s) from 0.05% to 20% by weight and better still from 0.1% to 10% of the total weight of this composition.

(C) Surfactants:

The surfactant(s) that may be present in the reducing composition may be chosen, without preference, from anionic, nonionic, amphoteric and cationic surfactants.

Anionic, nonionic, amphoteric or cationic surfactants that are suitable for use in the invention are especially the following:

Anionic Surfactants:

By way of example of anionic surfactants that may be used, alone or as mixtures, mention may be made of salts, in particular alkaline salts (sodium salts, magnesium salts, ammonium salts, amine salts, amino alcohol salts, etc.) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; (C₆-C₂₄) alkyl sulphosuccinates, (C₆-C₂₄)alkyl ether sulphosuccinates, (C₆-C₂₄)alkylamide sulphosuccinates; (C₆-C₂₄) alkyl sulphoacetates; (C₆-C₂₄)acyl sarcosinates; and (C₆-C₂₄)acyl glutamates.

Mention may also be made of (C₆-C₂₄)alkylpolyglycoside carboxylic esters such as alkylpolyglucoside citrates, alkylpolyglucoside tartrates, alkylpolyglucoside sulphosuccinates and alkylpolyglucoside sulphosuccinamates; acyl isethionates and N-acyl taurates, the alkyl or acyl radical of all of these different compounds preferably containing from 12 to 20 carbon atoms and the aryl radical preferably denoting a phenyl or benzyl group.

It is also possible to use fatty acid salts such as the salts of oleic, ricinoleic, palmitic and stearic acid, and the salts of coconut oil acid or hydrogenated coconut oil acid; acyl lactylates in which the acyl radical contains 8 to 20 carbon atoms; alkyl D-galactoside uronic acids and their salts; polyoxyalkylenated (C₆-C₂₄)alkyl ether carboxylic acids, polyoxyalkylenated (C₆-C₂₄)alkylamido ether carboxylic acids and their salts, in particular those comprising from 2 to 50 alkylene oxide groups and more especially ethylene oxide groups, and mixtures thereof.

Nonionic Surfactants:

Nonionic surfactants are compounds that are well known per se (see for example the “Handbook of Surfactants” by M. R. Porter, published by Blackie & Son (Glasgow and London), 1991, pp. 116-178) and their nature is not a critical feature in the context of the present invention.

Thus, used alone or as mixtures, they can be chosen especially from polyethoxylated and polypropoxylated, alkylphenols, α-diols or alcohols having a fatty chain containing, for example, 8 to 18 carbon atoms, it being possible for the number of ethylene oxide or propylene oxide groups to range in particular from 2 to 50; copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; oxyethylenated fatty acid esters of sorbitan having from 2 to 30 mol of ethylene oxide; fatty acid esters of sucrose, fatty acid esters of polyethylene glycol; alkylpolyglycosides; N-alkylglucamine derivatives, and amine oxides such as (C₁₀-C₁₄)alkylamine oxides or N-acylaminopropyl-morpholine oxides.

Amphoteric Surfactants:

The amphoteric (or zwitterionic) surfactants, the nature of which is not a critical feature in the context of the present invention, may be chosen especially, alone or as mixtures, from aliphatic secondary or tertiary amine derivatives in which the aliphatic radical is a linear or branched chain containing 8 to 18 carbon atoms and containing at least one water-solubilizing anionic group, for example carboxylate, sulphonate, sulphate, phosphate or phosphonate.

Mention may also be made of (C₈-C₂₀)alkylbetaines, sulphobetaines, (C₈-C₂₀)alkyl-amido(C₁-C₆)alkylbetaines or (C₈-C₂₀)alkylamido(C₁-C₆)-alkylsulphobetaines.

Among the amine derivatives, mention may be made especially of the products sold by the company Rhodia Chimie under the trade name Miranol®, which are described in U.S. Pat. No. 2,528,378 and U.S. Pat. No. 2,781,354 and classified in the CTFA dictionary, 5th edition, 1993, under the names “disodium cocoamphodiacetate”, “disodium lauroamphodiacetate”, “disodium caprylamphodiacetate”, “disodium capryloamphodiacetate”, “disodium cocoamphodipropionate”, “disodium lauroamphodipropionate”, “disodium caprylamphodipropionate”, “disodium capryloamphodipropionate”, “lauroamphodipropionic acid” and “cocoamphodipropionic acid”.

Cationic Surfactants:

As cationic surfactants that may be used, alone or as mixtures, mention may be made of salts of optionally polyoxyalkylenated primary, secondary or tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; and amine oxides of cationic nature.

When the reducing composition comprises one or more surfactants, then this or these agent(s) generally represent(s) from 0.01% to 40% by weight and better still from 0.1% to 30% of the total weight of this composition.

(D) Rheology Modifiers Other than the Polymers (B):

For the purposes of the present invention, the term “rheology modifier” means any compound capable of giving a viscosity to the reducing composition such that, once it is applied onto keratin fibres, this composition does not run, and remains perfectly localized at the point of application

It should be noted that said agent lacks a hydrophobic chain, i.e. a saturated or unsaturated, aromatic or non-aromatic, linear or branched C₈-C₃₀ hydrocarbon-based chain optionally comprising one or more oxyalkylene (oxyethylene and/or oxypropylene) units.

The rheology modifier(s) that may be present in the reducing composition is (are) polymers of natural origin or synthetic polymers, and are advantageously chosen from those conventionally used in cosmetics.

Examples of synthetic polymers that may be mentioned include polyvinylpyrrolidone, polyacrylic acid, polyacrylamide, non-crosslinked poly(2-acryl-amidopropanesulphonic acid) (Simugel EG from the company SEPPIC), crosslinked poly(2-acrylamido-2-methylpropanesulphonic acid), free or partially neutralized with ammonia (Hostacerin AMPS from Clariant), mixtures of non-crosslinked poly(2-acryl-amido-2-methylpropanesulphonic acid) with hydroxyalkyl-cellulose ethers or with poly(ethylene oxide)s, as described in patent U.S. Pat. No. 4,540,510; mixtures of poly((meth)acrylamido(C₁-C₄)alkylsulphonic acid), which is preferably crosslinked, with a crosslinked copolymer of maleic anhydride and of a (C₁-C₅)alkyl vinyl ether (Hostacerin AMPS/Stabileze QM from the company ISF).

The thickening polymers of natural origin are preferably polymers comprising at least one sugar unit, for instance nonionic guar gums, optionally modified with C₁-C₆ hydroxyalkyl groups; biopolysaccharide gums of microbial origin, such as scleroglucan gum or xanthan gum; gums derived from plant exudates, such as gum arabic, ghatti gum, karaya gum, gum tragacanth, carrageenan gum, agar gum and carob gum; pectins; alginates; starches; hydroxy (C₁-C₆)alkylcelluloses and carboxy (C₁-C₆)alkyl celluloses.

It should be noted that the term “sugar unit” denotes a monosaccharide (i.e. monosaccharide or oside or simple sugar) portion, an oligosaccharide portion (short chains formed from a sequence of monosaccharide units, which may be different) or a polysaccharide portion [long chains consisting of monosaccharide units, which may be different, i.e. polyholosides or polyosides]. The saccharide units may also be substituted with alkyl, hydroxyalkyl, alkoxy, acyloxy or carboxyl radicals, the alkyl radicals containing from 1 to 4 carbon atoms.

Examples of nonionic, unmodified guar gums that may be mentioned, inter alia, include Guargel D/15 (Noveon); Vidogum GH 175 (Unipectine), Meypro-Guar 50 and Jaguar C (Meyhall/Rhodia Chimie); and the modified nonionic guar gums that may be mentioned include Jaguar HP8, HP60, HP120, DC 293 and HP 105 (Meyhall/Rhodia Chimie); Galactasol 4H4FD2 (Aqualon).

The biopolysaccharide gums of microbial or plant origin are well known to those skilled in the art and are described especially in the book by Robert L. Davidson entitled “Handbook of Water soluble gums and resins” published by McGraw Hill Book Company (1980).

Among these gums, mention will be made of scleroglucans such as, especially, Actigum CS from Sanofi Bio Industries; Amigel from Alban Muller International, and also the glyoxal-treated scleroglucans described in FR 2 633 940); xanthan gums, for instance Keltrol, Keltrol T, Keltrol Tf, Keltrol Bt, Keltrol Rd, Keltrol Cg (Nutrasweet Kelco), Rhodicare S and Rhodicare H (Rhodia Chimie); starch derivatives, for instance Primogel (Avebe); hydroxy-ethylcelluloses such as Cellosize QP3L, QP4400 H, QP30000H, HEC30000A and Polymer PCG10 (Amerchol), Natrosol 250HHR, 250MR, 250M, 250HHXR, 250HHX, 250HR, HX (Hercules) and Tylose H1000 (Hoechst); hydroxy-propylcelluloses, for instance Klucel EF, H, LHF, MF and G (Aqualon); carboxymethylcelluloses, for instance Blanose 7M8/SF, refined 7M, 7LF, 7MF, 9M31F, 12M31XP, 12M31P, 9M31XF, 7H, 7M31, 7H3SXF (Aqualon), Aquasorb A500 (Hercules), Ambergum 1221 (Hercules), Cellogen HP810A, HP6HS9 (Montello) and Primellose (Avebe).

The composition may also comprise in replacement for or in combination with at least one rheology modifier, at least one linear or non-linear, saturated or unsaturated C₆-C₃₀ carboxylic acid alkylamide, and optionally bearing one or more hydroxyl groups.

Moreover, the nitrogen of the amide group may be monosubstituted or disubstituted. It is preferably monosubstituted.

The amide may comprise 1 to 20 oxyalkylene (oxyethylene and/or oxypropylene) units, preferably oxyethylene units.

When the reducing composition comprises one or more rheology modifiers, then this or these agent(s) generally represent(s) from 0.05% to 20% by weight and better still from 0.1% to 10% of the total weight of this composition.

(E) pH Modifiers:

The pH of the reducing composition may be between 1.5 and 12.

However, it is preferable for this pH to be between 1.5 and 10 and better still between 1.5 and 7 in the case where the reducing composition is intended for bleaching keratin fibres, and for it to be between 6 and 12 and preferably between 7 and 11 when the said composition is intended for permanently reshaping keratin fibres.

Such pH values may be obtained using acidifying or basifying agents.

As examples of acidifying agents that may be used, mention may be made of mineral or organic acids, for instance hydrochloric acid, phosphoric acid, orthophosphoric acid, acetic acid, tartaric acid, citric acid, lactic acid, boric acid and sulphonic acids.

As regards the basifying agents, it should be noted that the permanent reshaping of keratin fibres envisaged in the context of the invention is not a lanthionization. Consequently, the basifying agents are preferably chosen from compounds other than metal hydroxides and guanidine hydroxides, for instance aqueous ammonia, ammonium or alkaline carbonates, alkanolamines such as monoethanolamine, diethanolamine and triethanolamine and derivatives thereof, hydroxyalkylamines, oxyethylenated and/or oxypropylenated ethylenediamines, sodium hydroxide, potassium hydroxide and the compounds corresponding to formula (XIX) below: 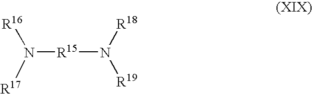
in which:
R¹⁵ is a propylene residue optionally substituted with a hydroxyl group or a C₁-C₄ alkyl radical; whereas
R¹⁶, R¹⁷, R¹⁸ and R¹⁹, which are identical or different, represent a hydrogen atom, a C₁-C₄ alkyl radical or a C₁-C₄ hydroxyalkyl radical.

When the reducing composition comprises one or more acidifying agents or one or more basifying agents, then this or these agent(s) generally represent(s) from 0.01% to 30% by weight relative to the total weight of this composition.

(F) Solvents:

The solvents that may be present in the reducing composition are especially water and mixtures composed of water and of one or more cosmetically acceptable organic solvents, this or these organic solvent(s) possibly being, in particular, alcohols such as ethanol, isopropanol, benzyl alcohol, phenylethyl or alcohol or cetyl alcohol, polyols, for instance propylene glycol and glycerol; glycol ethers, for instance ethylene glycol monomethyl ether, monoethyl ether and monobutyl ether, and also glycol alkyl ethers, for instance diethylene glycol monoethyl ether or monobutyl ether.

This or these organic solvent(s), when it is (they are) present in the reducing composition, generally represent(s) from 0.5% to 20% by weight and better still from 2% to 10% by weight relative to the total weight of this composition.

Depending on its intended use and the particular properties desired to be given thereto as a function of this use, the reducing composition may also comprise one or more adjuvants chosen from mineral or organic fillers such as silica or clays, binders such as vinylpyrrolidone, oils or waxes, polyalkylene glycols or polyalkylene glycol derivatives, lubricants such as polyol stearates or alkali metal or alkaline-earth metal stearates, antifoams, volatile or non-volatile, cyclic, linear or branched silicones, which are optionally modified, especially with amine groups, dyes, matting agents, for instance titanium oxides, preserving agents, and/or fragrances.

Each of these adjuvants may represent, when it is present in the reducing composition, up to 30% by weight relative to the total weight of this composition.

The reducing composition preferably comprises a complexing agent of formula (I) and a reductone and/or a sulphinate as reducing agent(s) in the case where it is intended for bleaching keratin fibres, whereas it preferably comprises a complexing agent and a thiol and/or a sulphite and/or a sulphinate as reducing agent(s), in the presence of a cationic polymer chosen from the abovementioned cationic polymers in the case where it is intended for permanently reshaping keratin fibres.

A subject of the invention is also a process for bleaching or permanently reshaping keratin fibres, comprising the steps consisting in:

a) applying a reducing composition as defined above to keratin fibres;

b) leaving the reducing composition to stand on the keratin fibres for a time that is sufficient to obtain the desired bleaching or permanent reshaping;

c) rinsing the keratin fibres to remove the reducing composition therefrom;

d) washing the keratin fibres one or more times, rinsing them after each wash, and optionally drying them;

said process also comprising, between steps c) and d), in the case of a permanent reshaping, the steps consisting in: i) applying an oxidizing composition, for example based on oxygen peroxide, to the keratin fibres, ii) leaving the oxidizing composition to stand on the keratin fibres for a time that is sufficient to obtain the fixing of the desired reshaping; and iii) rinsing the keratin fibres with water to remove the oxidizing composition therefrom.

In step b), the time for which the reducing composition is left to stand on the keratin fibres may range from 1 to 60 minutes but is preferably between 10 and 45 minutes, whereas, in step ii), the standing time of the oxidizing composition on the keratin fibres is from about 1 to 20 minutes and preferably from 1 to 10 minutes.

In the case of a permanent-reshaping operation, mechanical means for placing the keratin fibres under tension, such as rollers, may be used before, during or after application of the reducing composition, and may be removed before or after rinsing out the oxidizing composition.

A subject of the invention is also a device or “kit” for bleaching keratin fibres, comprising at least two compositions A and B intended to be mixed together to obtain a ready-to-use reducing composition, said device being characterized in that at least one of the compositions A and B contains one or more reducing agents and at least one of the compositions A and B contains one or more compounds corresponding to the general formula (I) below:
R—N—(CH(R′)CO₂X)₂ (I)
in which:
R represents a hydrogen atom or a group —CH(CO₂X)—(CH₂)₂CO₂X, —CH₂—CH₂—OH, —CH(CH₃)—CO₂X or —(CH₂)₂—N(COR″)—CH₂—CO₂X;
R″ represents a linear or branched alkyl group containing from 1 to 30 carbon atoms, or a cycloalkyl group containing from 3 to 30 carbon atoms;
R′ represents a group —CH₂CO₂X when R represents a hydrogen atom, whereas R′ represents a hydrogen atom when R is other than a hydrogen atom; and
X represents a hydrogen atom or a monovalent or divalent cation derived from an alkali metal, from an alkaline-earth metal, from a transition metal or from an organic amine, or an ammonium cation.

A subject of the invention is also a device or “kit” for permanently reshaping keratin fibres, comprising, firstly, either a composition A or at least two compositions A′ and B′ intended to be mixed together to obtain a ready-to-use reducing composition, and, secondly, a ready-to-use oxidizing composition C or at least two compositions D and E intended to be mixed together to obtain a ready-to-use oxidizing composition, said device being characterized in that either composition A or at least one of the compositions A′ and B′ contains one or more reducing agents, and either composition A or at least one of the compositions A′ and B′ contains at least one or more compounds corresponding to the general formula (I) below:
R—N—(CH(R′)CO₂X)₂ (I)
in which:
R represents a hydrogen atom or a group —CH(CO₂X)—(CH₂)₂CO₂X, —CH₂—OH₂—OH, —CH(CH₃)—CO₂X or —(CH₂)₂—N(COR″)—CH₂—CO₂X;
R″ represents a linear or branched alkyl group containing from 1 to 30 carbon atoms, or a cycloalkyl group containing from 3 to 30 carbon atoms;
R′ represents a group —CH₂CO₂X when R represents a hydrogen atom, whereas R′ represents a hydrogen atom when R is other than a hydrogen atom; and
X represents a hydrogen atom or a monovalent or divalent cation derived from an alkali metal, from an alkaline-earth metal, from a transition metal or from an organic amine, or an ammonium cation.

Whether the devices are intended for bleaching or permanent reshaping, compositions A, A′ and B may be solutions or one may be in the form of a powder or a cream and the other in the form of an aqueous composition.

Furthermore, a subject of the invention is the use of a reducing composition, of a process for bleaching or permanently reshaping or of a device as defined above, for bleaching or permanently reshaping human keratin fibres and more especially the hair.

The use of the compounds of formula (I) as complexing agents in compositions for bleaching or permanently reshaping keratin fibres, and especially hair fibres, has many advantages.

Specifically, not only do these compounds show noteworthy complexing properties with respect to metal cations and thus considerably reduce the risk of the bleaching or permanent-reshaping operation being accompanied by adverse effects such as breaking of the hair or burning of the scalp, but also it is found that they are highly soluble in water and stable in aqueous medium, compatible with all the compounds liable to be included in the constitution of hair reducing compositions, very well tolerated by the skin, biodegradable and relatively inexpensive.

Besides the preceding arrangements, the invention also comprises other arrangements which will emerge from the rest of the description that follows, which refer to embodiments of reducing compositions for, on the one hand, bleaching and, on the other hand, for permanently reshaping keratin fibres.

It goes without saying that these examples are given for illustrative purposes and do not in any way limit the subject of the invention.

### DETAILED DESCRIPTION OF SPECIFIC EMBODIMENTS

### EXAMPLE 1

### Ready-to-Use Reducing Compositions for Bleaching Keratin Fibres

Three ready-to-use reducing compositions—A, B and C, respectively—for bleaching keratin fibres were prepared. Their qualitative and quantitative composition is given in Table I below, in which the amounts of the various constituents are expressed in grams.

TABLE I

| Constituents | A | B | C |
|---|---|---|---|
| Sodium hydroxymethane sulphinate | 7 | 7 | — |
| Ascorbic acid | — | — | 10 |
| Sodium sulphite | — | — | 3 |
| Cetyl alcohol | 3 | 3 | 3 |
| Sodium lauryl sulphate | 0.7 | 0.7 | 0.5 |
| Benzyl alcohol | 2 | 2 | 2 |
| Propylene glycol | 10 | 10 | 10 |
| Aqueous 40% solution of disodium | — | 0.13 | 0.07 |
| salt of 2-hydroxyethylimino | | | |
| diacetic acid* | | | |
| Aqueous 40% solution of | 0.15 | — | — |
| trisodium salt of methylglycine | | | |
| diacetic acid** | | | |

| | |
|---|---|
| Aqueous 85% H₂PO₄ solution | qs pH = 2.7 |
| Water | qs 100 g |

*XUS-40855.00 - from Dow Chemical
**Trilon ® M Liquid - from BASF

Compositions A, B and C were applied, in a bath ratio of 10 g of composition per 1 g of hair, to naturally grey hair (containing 90% white hairs) dyed beforehand with the shade 6.66 from the L'Oréal Majirouge® range.

After a leave-in time of 30 minutes, the hair was rinsed thoroughly with water and then treated with aqueous 3% H₂O₂ solution for 2 minutes, after which the hair was again rinsed thoroughly with water. The hair was then washed with a standard shampoo and then dried with a hair dryer.

Strong, uniform bleaching of the hair treated with compositions A, B and C was observed. Specifically in the three cases, the strong red glints imparted by the dye almost totally disappeared, again leaving the hair almost as it was before having undergone this dyeing.

### EXAMPLE 2

### Ready-to-Use Reducing Compositions for Permanently Reshaping Keratin Fibres

Three ready-to-use reducing compositions—D, E and F, respectively—for permanently reshaping keratin fibres were prepared. Their qualitative and quantitative composition is given in Table II below, in which the amounts of the various constituents are expressed in grams.

TABLE II

| Constituents | D | E | F |
|---|---|---|---|
| Thioglycolic acid | 9.2 | 9.2 | 9.2 |
| Aqueous 20% NH₃ solution | 9.3 | 9.3 | 9.3 |
| Ammonium carbonate | 4.5 | 4.5 | 4.5 |
| Aqueous 30% solution of | 1.3 | 1.3 | 1.3 |
| cocoylamidopropylbetaine/glyceryl | | | |
| Monolaurate (25:5) | | | |
| Aqueous 60% solution of a cationic | 1.7 | 1.7 | 1.7 |
| polymer of formula W | | | |
| Aqueous 40% solution of disodium | — | — | 0.05 |
| salt of 2-hydroxyethylimiodiacetic | | | |
| acid* | | | |
| Aqueous 40% solution of trisodium | 0.15 | — | — |
| salt of methylglycine diacetic | | | |
| acid** | | | |
| Sodium iminodisuccinate*** | — | 1 | — |

| | |
|---|---|
| Water | qs 100 g |

*XUS-40855.00 - from Dow Chemical
**Trilon ® M Liquid - from BASF
***Iminodisuccinate VP OC sodium salt powder (N-305) - from Bayer

Compositions D, E and F were applied to moistened hair rolled up beforehand on rollers 9 mm in diameter.

After a leave-in time of 15 minutes, the hair was rinsed thoroughly with water and then treated with an 8-volumes aqueous H₂O₂ solution of pH 3, for 5 minutes, after which the hair was again rinsed thoroughly with water.

The rollers were then removed and the hair was dried.

This hair, whether it had been treated with composition D, composition E or composition F, had beautiful uniform curls.

1. A reducing composition for bleaching or permanently reshaping keratin fibres, comprising:
a) at least one reducing agent, and
b) at least one compound of formula (I):
R—N—(CH(R′)CO₂X)₂ (I)
wherein:
R is a hydrogen atom or a —CH(CO₂X)—(CH₂)₂CO₂X, —CH₂—CH₂—OH, —CH(CH₃)—CO₂X or —(CH₂)₂—N(COR″)—CH₂—CO₂X group;
R″ is a linear or branched alkyl group containing from 1 to 30 carbon atoms, or a cycloalkyl group containing from 3 to 30 carbon atoms;
R′ is a —CH₂CO₂X group when R is a hydrogen atom, or a hydrogen atom when R is other than a hydrogen atom; and
X is a hydrogen atom or a monovalent or divalent cation chosen from an alkali metal, alkaline-earth metal, transition metal, organic amine or an ammonium cation.

2. The composition according to claim 1, wherein said monovalent or divalent cation is an alkali metal cation, an alkaline-earth metal cation, a divalent transition metal cation or a monovalent cation chosen from an organic amine or an ammonium cation.

3. The composition of claim 1, wherein said compound of formula (I) is methylglycine diacetic acid, 2-hydroxyethylimino diacetic acid, N-lauroyl-N,N′,N′-ethylenediamine triacetic acid, iminodisuccinic acid or N,N-dicarboxymethyl-L-glutamic acid, an alkali metal salts thereof, an alkaline-earth metal salt thereof, a transition metal salt thereof, or a mixture thereof.

4. The composition of claim 1, wherein said compound of formula (I) is 2-hydroxyethylimino diacetic acid or methylglycine diacetic acid or a sodium salt thereof, or a mixtures thereof.

5. The composition of claim 1, wherein said compound of formula (I) is present in an amount of from 0.001% to 10% by weight relative to the total weight of said composition.

6. The composition of claim 5, wherein said compound of formula (I) is present in an amount of from 0.001 to 5% by weight relative to the total weight of said composition.

7. The composition of claim 1, wherein said reducing agent is a reductone or a salt or an ester thereof, or a sulphite or a sulphinate.

8. The composition of claim 1, said reducing agent is thiol or a salt or ester thereof, or sulphite or sulphinate.

9. The composition of claim 8, wherein said reducing agent is thioglycolic acid, thiolactic acid, cysteamine or cysteine, or a salt or ester thereof.

10. The composition of claim 1, wherein said reducing agent is present in an amount of from 0.1% to 30% by weight relative to the total weight of said composition.

11. The composition of claim 10, wherein said reducing agent is present in an amount of from 0.5% to 20% by weight relative to the total weight of said composition.

12. The composition of claim 1, further comprising a cationic or amphoteric conditioning polymer.

13. The composition of claim 1, further comprising an amphiphilic polymer which is nonionic, anionic, cationic, or amphoteric, wherein said amphiphilic polymer comprises a hydrophobic chain.

14. The composition of claim 1, further comprising a surfactant.

15. The composition of claim 1, further comprising a rheology modifier other than the amphiphilic polymers of claim 13.

16. The composition of claim 1, further comprising an acidifying or basifying agent.

17. The composition of claim 1, further comprising a solvent.

18. The composition of claim 1, further comprising an adjuvant. chosen from a mineral or organic filler, binder, lubricant, antifoam, silicone, dye, matting agent, preserving agent or fragrance.

19. A method of bleaching keratin fibres, comprising the steps of:
a) applying to the keratin fibres the reducing composition of claims 1;
b) leaving the reducing composition to stand on the keratin fibres for a sufficient time to obtain the desired bleaching;
c) rinsing said keratin fibres to remove the reducing composition therefrom;
d) washing said keratin fibres one or more times, rinsing said keratin fibres after each wash.

20. A “kit” for bleaching keratin fibres, comprising at least two compositions A and B intended to be mixed together to obtain a ready-to-use reducing composition, wherein,
a) at least one of the compositions A and B contains at least one reducing agent, and
b) at least one of the compositions A and B contains at least one compounds of formula (I):
R—N—(CH(R′)CO₂X)₂ (I)
wherein:
R is a hydrogen atom or a —CH(CO₂X)—(CH₂)₂CO₂X, —CH₂—CH₂—OH, —CH(CH₃)—CO₂X or —(CH₂)₂—N(COR″)—CH₂—CO₂X group;
R″ is a linear or branched alkyl group containing from 1 to 30 carbon atoms, or a cycloalkyl group containing from 3 to 30 carbon atoms;
R′ is a —CH₂CO₂X group when R is a hydrogen atom, or a hydrogen atom when R is other than a hydrogen atom; and
X is a hydrogen atom or a monovalent or divalent cation chosen from an alkali metal, an alkaline-earth metal, a transition metal, an organic amine or an ammonium cation.

21. A “kit” for permanently reshaping keratin fibres, comprising:
a) either a ready-to-use reducing composition A or at least two compositions A′ and B′ intended to be mixed together to obtain a ready-to-use reducing composition, and,
b) a ready-to-use oxidizing composition C or at least two compositions D and E intended to be mixed together to obtain a ready-to-use oxidizing composition, wherein,
either said composition A or at least one of said compositions A′ and B′ contains at least one reducing agent, and
either said composition A or at least one of said compositions A′ and B′ contains at least one compound of formula (I):
R—N—(CH(R′)CO₂X)₂ (I)
wherein:
R is a hydrogen atom or a —CH(CO₂X)—(CH₂)₂CO₂X, —CH₂—CH₂—OH, —CH(CH₃)—CO₂X or —(CH₂)₂—N(COR″)—CH₂—CO₂X group;
R″ is a linear or branched alkyl group containing from 1 to 30 carbon atoms, or a cycloalkyl group containing from 3 to 30 carbon atoms;
R′ is a —CH₂CO₂X group when R is a hydrogen atom, or a hydrogen atom when R is other than a hydrogen atom; and
X represents a hydrogen atom or a monovalent or divalent cation chosen from an alkali metal, an alkaline-earth metal, a transition metal, an organic amine or an ammonium cation.

22. (canceled).

23. The composition of claim 12, wherein said cationic or amphoteric conditioning polymer is present in an amount of from 0.01% to 10% by weight relative to the total weight of said composition.

24. The composition of claim 23, wherein said cationic or amphoteric conditioning polymer is present in an amount of from 0.05% to 5% by weight relative to the total weight of said composition.

25. The composition of claim 13, wherein said amphiphilic polymer is present in an amount of from 0.05% to 20% by weight relative to the total weight of said composition.

26. The composition of claim 25, wherein said amphiphilic polymer is present in an amount of from 0.1% to 10% by weight relative to the total weight of said composition.

27. The composition of claim 14, wherein said surfactant is present in an amount of from 0.01% to 40% by weight relative to the total weight of said composition.

28. The composition of claim 27, wherein said surfactant is present in an amount of from 0.1% to 30% by weight relative to the total weight of said composition.

29. The composition of claim 15, wherein said rheology modifier is present in an amount of from 0.05% to 20% by weight relative to the total weight of said composition.

30. The composition of claim 29, wherein said rheology modifier is present in an amount of from 0.1% to 10% by weight relative to the total weight of said composition.

31. The composition of claim 16, wherein said acidifying or basifying agent is present in an amount of from 0.01% to 30% by weight relative to the total weight of said composition.

32. The composition of claim 17, wherein said solvent is water or a mixture composed of water and a cosmetically acceptable organic solvent.

33. The composition of claim 32, wherein said solvent is present in an amount of from 0.5% to 20% by weight relative to the total weight of said composition.

34. The composition of claim 33, wherein said solvent is present in an amount of from 2% to 10% by weight relative to the total weight of said composition.

35. The method of claim 19, further comprising the step of drying said keratin fibres.

36. A method of permanently reshaping keratin fibres, comprising the steps of:
a) applying to said keratin fibres the reducing composition of claim 1;
b) leaving said reducing composition on said keratin fibres for a sufficient time to obtain the desired reshaping;
c) rinsing said keratin fibres to remove said reducing composition therefrom;
d) applying an oxidizing composition to said keratin fibres;
e) leaving said oxidizing composition on said keratin fibres for a sufficient time to obtain the desired reshaping;
f) rinsing said keratin fibres with water to remove said oxidizing composition therefrom;
g) washing said keratin fibres one or more times, rinsing them after each wash.

37. The method of claim 36, further comprising the step of drying said keratin fibres.
